(19) United States
(12) Patent Application Publication
(10) Pub. No.: US 2022/0351820 A1
(43) Pub. Date: Nov. 3, 2022

(54) Method for determining glucose-dependent insulin sensitivity factors

(71) Applicant: Christopher Richard Wilson, Oceanside, CA (US)

(72) Inventor: Christopher Richard Wilson, Oceanside, CA (US)

(73) Assignee: Christopher Richard Wilson, Oceanside, CA (US)

(21) Appl. No.: 17/246,626

(22) Filed: May 1, 2021

(51) Int. Cl. G16H 20/17; A61M 5/172

(52) U.S. Cl. CPC G16H 20/17; A61M 5/1723; A61M 2205/50; A61M 2230/201

(57) ABSTRACT

This invention discloses a method of calculating insulin sensitivity factors for use in calculating insulin doses dynamically as a function of current blood glucose level based on the patient's total daily insulin dose. When the total daily insulin is inserted into the equation, the result is a mathematical function that can be used to estimate the effects of each unit of insulin delivered on a patient's blood glucose level at their current blood glucose level. This mathematical equation is valid over a wide range of diabetic conditions and could be widely used throughout the world at very little cost (and at considerable improvement) to the current technology.

### CROSS REFERENCE TO RELATED APPLICATIONS

None.

### STATEMENT REGARDING FEDERALLY SPONSORED RESEARCH OR DEVELOPMENT

This invention was not federally sponsored.

### INVENTOR

Christopher Richard Wilson, citizen of Oceanside, California, and citizen of USA.

### ATTORNEY DOCKET

CWilson-PP-1

### BACKGROUND OF THE INVENTION

### Field Of The Invention

This invention relates to a general field of medical medication calculations, and more specifically, to a method by which the insulin sensitivity factors can be calculated to determine correction doses of insulin for patients with varying levels of glycemia.

### BRIEF DESCRIPTION OF INVENTION

The invention is a formula and method of use of the formula to determine how insulin sensitivity factors relate to correct, and correction, doses of insulin. The invention is relevant to a variety of human patients with varying levels of glycemia.

### STATEMENT OF THE PROBLEM SOLVED

Diabetes is one of the most prevalent and serious diseases facing modern humans. It is a chronic health issue that affects how a person turns food into energy. As food is digested, most of it turns into glucose, a “sugar”, and is released into the person's bloodstream. The hormone insulin acts as a stop-value to let in the correct amount of “blood sugar” into the body at any given time and under given conditions. A diabetic person either doesn't make enough insulin or does not use its insulin efficiently, resulting in too high of a concentration of blood sugar in the bloodstream. This can lead to serious health problems such as heart and kidney diseases and vision impairment/loss.

It is estimated by the Center for Disease Control that at least 33% of adults in the United States have Prediabetes, and over 10% have actual diabetes. It is estimated that nearly half a billion people in the world have diabetes, and this number is expected to double in the next 40 or 50 years. Thus, helping diabetic people is extremely important.

The current state-of-the-art in Diabetes care is a process known as automated insulin delivery. These systems operate by receiving data from a small, wearable monitor which measures interstitial blood glucose, and a pump system that delivers insulin to the patient. The pump systems run on “black box” software and are based on proprietary algorithms developed or licensed by individual insulin pump manufacturers.

However, insulin sensitivity varies for individual patients according to the concentration of glucose in human blood at the time. At higher levels of blood glucose, insulin sensitivity is reduced, resulting in patients and automated insulin delivery systems dosing insufficient amounts of insulin to correct the hyperglycemia.

This “black box” industry standard creates a serious problem in that the current “modern” technology is based on an incorrect assumption that is relied on by the entire industry. For over 30 years, settings for insulin pumps have consisted of 3 configurable settings:
1. A “basal rate” of insulin that is (ideally) delivered continuously to support basic metabolic needs in the absence of food or other external factors.
2. A “carb ratio” that defines for the pump the mass of carbohydrate eaten that is disposed of by each unit of insulin given in addition to the basal rate.
3. A “correction factor” or “insulin sensitivity factor” (ISF) which defines a static value by which a unit of insulin is assumed to reduce a patient's blood glucose level.

The incorrect assumption that all current insulin delivery systems make is that the insulin sensitivity factor, or ISF, is a static value. It is not. ISF declines linearly at approximately 5 (five) milligrams per deciliter per unit of insulin for each 50 (fifty) milligrams per deciliter that the patient's blood glucose level is above a value of (roughly) 100 mg/dL. This results in patients being both over- and under-dosed by their insulin pumps, depending on their blood glucose level. It also means that current systems do not correctly account for the effects of insulin that has already been administered in predicting and preventing hypoglycemia.

### SOLUTION PRESENTED BY THIS INVENTION

The current invention that is described more fully in this patent application is a mathematical formula, and the use thereof, which corrects the current state-of-the-art erroneous calculations. Through identification, assessment of use of these calculations, the invention corrects those calculations, allowing more accurate dosing and more accurate predictions of the effect of insulin already administered to the patient.

### IMPROVEMENTS PROVIDED BY THIS INVENTION: DESCRIPTION ON HOW THE INVENTION IS AN IMPROVEMENT OVER EXISTING TECHNOLOGY

Current insulin dosage calculations, whether done by humans or automatically by automated insulin delivery algorithms, assume a static number for the “Insulin sensitivity factor” (ISF), which is the reduction in blood glucose estimated to result from the administration of one unit of insulin. This invention provides a mechanism for making that ISF vary relative to the patient's current blood glucose concentration. Individual and business demographics that would use this Invention: •Persons living with insulin-requiring Diabetes. •Physicians and other healthcare professionals who calculate insulin doses and ratios for patients to apply. •Companies designing, manufacturing, marketing, and distributing automated glycemic controller software as a component of automated insulin delivery systems.

### DESCRIPTION OF THE BENEFITS OF THIS INVENTION TO ITS USERS

More accurate insulin sensitivity estimations will allow for more accurate dose calculation in automated insulin delivery systems. Additionally, patients calculating individual correction doses of insulin using this method are less likely to under- or over-dose their insulin, reducing glycemic variability and improving the amount of time spent in the optimal glucose range.

### METHODS USED TO OBTAIN READINGS

To obtain the various numbers required for use in this equation, a variety of techniques/machines are used. In case the reader is not experienced in diabetic issues, readings used as inputs into this formula are obtained from either a “fingerstick glucometer” that measures glucose levels in whole blood via dynamic electrochemistry using either glucose oxidase or glucose dehydrogenase, or via a continuous glucose monitor (CGM) that provides estimates of blood glucose levels every 5 minutes.

There are currently two real-time CGM systems in the US which have been approved by the FDA to have their measurements used as a basis for dosing insulin (considered a “high-risk” medication): the Dexcom G6, and the Eversense system from Senseonics. Additionally, while not a real-time CGM system, the Freestyle Libre from Abbott is also approved for use in dosing decisions without a confirmatory fingerstick, but it lacks the ability to “push” data to a connected pump, instead relying on the user to periodically “scan” the sensor with a phone or receiver to obtain a history of readings.

Thus there has existed a long-felt need for a method by which more accurate insulin sensitivity estimations can be obtained for use in existing software programs, such that a more accurate dosage of insulin can be provided to diabetic patients, and that this dosage can change over time depending on changing values of insulin sensitivity. The result, for any insulin monitor that uses the formula, would be a more accurate delivery of insulin with the resulting superior health benefits to the patient.

The current invention provides just such a solution by having a dynamic formula that takes into account previously obtained measurements, but combines them in a new formulation to produce an insulin sensitivity reading that can be incorporated into existing monitors and software to produce superior delivery of insulin to a diabetic patient.

### OBJECTS OF THE INVENTION

It is therefore an object of the present invention to provide a formula the provides an accurate insulin sensitivity reading.

Another object of the invention is to provide an insulin sensitivity reading that dynamically changes as the variables that make up the equation change.

Additional objects of the invention include providing a formula that will result in more accurate delivery of insulin.

A further object of the invention to for use in monitoring systems for hospitalized patients in that it can better predict hypoglycemia, and can be used to establish an absolute maximum blood glucose level beyond which the patient is likely to enter a condition known as Diabetic Ketoacidosis and/or Hyperglycemic Hyperosmolar Syndrome.

Another object of the invention is to function in a stand-alone monitoring system that monitors continuous glucose. The latest standalone CGM systems upload data to computer systems run by the manufacturers for analysis and reporting. At least one of these systems then attempts to predict glycemic excursions (blood glucose values outside the “target range” of 80-180 mg/dL) based on that history. This invention suggests a method that can be used to improve the predictions by those systems, as it relies not on historical trends but mathematical deductions based on the patient's current glycemic state and the amount of insulin that has been delivered to the patient over the past 5 hours.

A final object of the invention is to provide a method by which existing insulin monitors/pumps can incorporate this formula into the software and thereby improve their accuracy.

### STATEMENT OF THE INVENTION

Accordingly, one aspect the invention includes a formula for accurately calculating insulin sensitivity in a patient.

Preferably, the ISF (Insulin Sensitivity) is the sum of the BG (Glucose concentration measured in milligrams per deciliter) multiplied by negative 1/10, plus the number 2,300 divided by the TDD, which is the current average daily dose of insulin given to a specific patient.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1 is a formula, according to a preferred form of the invention.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

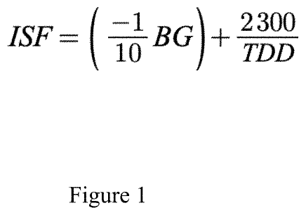

FIG. 1 is a formula, according to a preferred form of the invention.

### DEFINITION TERMS

ISF=Insulin Sensitivity

BG=Glucose concentration (measured in milligrams per deciliter).

TDD=the current average daily dose of insulin given to a specific patient.

To use this method of calculating insulin sensitivity, the patient's current average total daily dose (TDD) of insulin is inserted as the denominator of the static constant in the mathematical function at the spot labelled “TDD.” Blood glucose is then measured with a glucometer or other FDA-approved method of determining blood glucose, and the blood glucose concentration as expressed in milligrams per deciliter inserted at the spot labeled “BG.” Estimated current insulin sensitivity is then returned by evaluation of the function. (Chris, where does the 2300 come from?)

### UNIQUE FEATURES OF THE INVENTION

Current dose calculations for insulin use a static number as an estimate of insulin sensitivity, independent of glycemia. The method described provides a way to replace that static number used in dosage calculations with a mathematical function capable of computing dynamic insulin sensitivity across a wide glycemic range, allowing for more accurate dosing of insulin in patients with insulin-requiring Diabetes, resulting in significantly more accurate dosing during times of elevated blood glucose.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A method of measuring insulin sensitivity, comprising: a formula as follows:
$$ISF = ( \frac{- 1}{10} BG ) + \frac{2300}{TDD}$$
where,
ISF=Insulin Sensitivity
BG=Glucose concentration (measured in milligrams per deciliter).
TDD=the current average daily dose of insulin given to a specific patient.

2. The method of claim 1, where a user of the invention incorporates the formula into an item of existing software, where the item of existing software is used to run an insulin monitor and an insulin pump, where the item of existing software determines a dosage of insulin to be supplied to a patient.

3. The method of claim 1, where a user of the invention incorporates the formula into an item of new software, where the item of new software is used to run an insulin monitor and an insulin pump, where the item of existing software determines a dosage of insulin to be supplied to a patient.

4. The method of claim 1, where a user of the invention incorporates the formula into both a new software system and an existing software system, where both the new software system and the existing software system determines a dosage of insulin to be supplied to a patient.

5. The method of claim 1, where the formula the provides an accurate insulin sensitivity reading.

6. The method of claim 5, where the insulin sensitivity reading is dynamic in nature as one or more of various components of the equation change.

7. The method of claim 6, where the method can be used for monitoring systems for hospitalized patients to better predict hypoglycemia.

8. The method of claim 7, where the method can establish an absolute maximum blood glucose level beyond which the patient is likely to enter a condition known as Diabetic Ketoacidosis and/or Hyperglycemic Hyperosmolar Syndrome.

9. The method of claim 7, where the method can be used in a stand-alone monitoring system that monitors continuous glucose.

10. The method of claim 7, where the method can be used in existing insulin monitors/pumps and thereby improve their accuracy.

11. The method of claim 1, where the data range is 1,500 to 3,000.

12. The method of claim 1, where the data range is 2,000 to 2,500.

13. The method of claim 1, where the data range is 2,250 to 2,350.
